(19) United States
(12) Patent Application Publication
(10) Pub. No.: US 2008/0255653 A1
(43) Pub. Date: Oct. 16, 2008

(54) Multiple Stent Delivery System and Method

(75) Inventor: Daniel Schkolnik, Cotati, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/734,885

(22) Filed: Apr. 13, 2007

(51) Int. Cl. A61F 2/84

(52) U.S. Cl. 623/1.11

(57) ABSTRACT

A method of deploying multiple stents using a multiple stent delivery system includes moving a distal stent into contact with a stent-holding surface of a compressible expanded tip of an inner member. A sheath is retracted relative to the inner member to deploy the distal stent by holding the distal stent stationary with the inner member and retracting the sheath. The sheath is advanced relative to the inner member to reposition a proximal stent to capture, be positioned behind a next stent to be deployed by being compressed and passing the compressible expanded tip through the next proximal stent. The sheath is retracted relative to the inner member to bring the next stent to a pre-deployment position and then deployment of the next proximal stent can take place as already performed for the first deployed stent.

### BACKGROUND OF THE INVENTION

1. Field of the Invention

This invention relates generally to medical devices and procedures, and more particularly to a method and system of deploying stents in a vascular system.

2. Description of Related Art

Prostheses for implantation in blood vessels or other similar organs of the living body are, in general, well known in the medical art. For example, “self-expanding” stents are stents inserted into the vascular system in a compressed or contracted state, and permitted to expand upon removal of a restraint. Self-expanding stents typically employ a wire or tube configured (e.g., bent or cut) to provide an outward radial force and employ a suitable elastic material such as stainless steel or Nitinol (nickel-titanium). Nitinol may additionally employ shape memory properties.

A self-expanding stent is typically sized to be configured in a tubular shape of a slightly greater diameter than the diameter of the blood vessel in which the stent is intended to be used. In general, stents are typically deployed using a minimally invasive intraluminal delivery, i.e., cutting through the skin to access a lumen or vasculature or percutaneously via successive dilatation, at a convenient (and less traumatic) entry point, and routing the stent through the lumen to the site where the prosthesis is to be deployed.

Intraluminal deployment in one example is effected using a delivery catheter with coaxial inner tube, sometimes called the plunger, and sheath, arranged for relative axial movement. The stent is compressed and disposed within the distal end of the sheath in front of the inner tube.

The catheter is then maneuvered, typically routed though a lumen (e.g., vessel), until the end of the catheter (and the stent) is positioned in the vicinity of the intended treatment site. The inner tube is then held stationary while the sheath of the delivery catheter is withdrawn. The inner tube prevents the stent from moving back as the sheath is withdrawn.

As the sheath is withdrawn, the stent is gradually exposed from a distal end to a proximal end of the stent, the exposed portion of the stent radially expands so that at least a portion of the expanded portion is in substantially conforming surface contact with a portion of the interior of the lumen, e.g., blood vessel wall.

Lesions in the peripheral vasculature are sometimes considerably longer than those in the coronary arteries. To accommodate the greater length of the lesion, long stents are used, e.g., 150 mm or greater length stents.

However, the manufacturing equipment used to manufacture the long stents is typically larger than the manufacturing equipment used to manufacture short stents. Accordingly, long stents require a greater capital investment for the larger manufacturing equipment than small stents thus increasing the cost of manufacturing the long stents.

Further, long stents require more material and labor to manufacture than short stents. Thus, in the event that a long stent is defective, the cost of scrapping the long stent is greater than the cost of scrapping a short stent.

Further, to deploy a long stent a physician must overcome a greater amount of frictional force due to the greater total radial force between the long stent and the inside of the sheath constraining the long stent than in the case of a short stent. This can make accurate placement of the long stent more difficult.

In addition, the need to overcome a greater amount of frictional force when using a long stent places fundamental restrictions on the configuration, sizing, and materials to be used which can affect the degree to which the delivery profile of the delivery system used to deliver the long stent can be minimized. Having a large delivery profile affects the range of anatomical variation in which the long stent can be used.

For purposes of clarity of discussion, as used herein, the distal end of the catheter and of the stent is the end that is farthest from the operator (the end furthest from the handle) while the proximal end of the catheter and of the stent is the end nearest the operator (the end nearest the handle). However, those of skill in the art will understand that depending upon the access location, the stent and delivery system description may be consistent or opposite in actual usage.

### SUMMARY OF THE INVENTION

A method of deploying multiple stents using a multiple stent delivery system includes moving a distal stent into contact with a stent-pushing surface of a compressible expanded tip of an inner member. A sheath is retracted relative to the inner member to deploy the distal stent by holding the distal stent with the inner member fixed while retracting the sheath. The sheath is advanced relative to the inner member to reposition an end of an expanded tip of the middle member to a next stent stop position. As the sheath is pushed distally the expanded tip of the inner member is constrained slightly as it passes compressible expanded through the next proximal stent and returns to the full diameter of the inside of the sheath once it passes through the stent. The middle member and sheath are moved relative to one another to move the next stent (most distal stent in the sheath at that time) to a pre-deployment position, just adjacent to the end of the sheath, from where it can be predictably deployed. The sheath is retracted relative to the inner member to deploy the stent which has been already positioned at the pre-deployment position by holding the inner member stationary while retracting the sheath. Each stent is deployed one at a time, and the amount of frictional resistance force that needs to be overcome when using this configuration, is equal to the force needed to move only one stent within the sheath and not multiple stents simultaneously as is the case in the prior art.

In accordance with this example, the next proximal and distal stents are relatively short, e.g., 75 mm or less. More particularly, instead of using one long stent, several short stents including the next proximal and distal stents are used.

A physician manipulating the middle member and sheath must exert a force to overcome a lesser amount of frictional resistance force associated with moving a short stent (which exerts the radial force on the sheath proportional to its length) within the sheath compared to the higher frictional resistance force needed to move a longer (or multiple stents) with a frictional resistance force proportionally higher, the frictional resistance force expected to be proportional to the stent lengths that are being moved simultaneously at any one time. When manipulating long stent lengths, the higher compressive and tensile stresses placed on the middle member and sheath respectively, to overcome the larger frictional resistance forces associated with long stent lengths, make manipulation of the sheath and stent delivery more difficult than for shorter stents where such forces would be less. Thus making placement (movement) of the short stent lengths easier to perform and results in a more accurate stent deployment.

In addition, the lesser amount of frictional force of the short stent on the sheath allows the delivery profile of the multiple stent delivery system to be minimized. The reduced forces to be carried by the middle member and the sheath (by moving a short length of stent one at a time) allow their cross sections to be thinner than if they needed to be sized to carry the forces needed to overcome the larger frictional resistance associated with moving long stent lengths simultaneously. Minimizing the delivery profile maximizes the anatomical variation in which the multiple stent delivery system can be used.

These and other features according to the present invention will be more readily apparent from the detailed description set forth below taken in conjunction with the accompanying drawings.

### BRIEF DESCRIPTION OF THE DRAWINGS

FIG. 1 is a partial cross-sectional view of a multiple stent delivery system in accordance with one embodiment of the present invention;

FIG. 2 is a cross-sectional view of the multiple stent delivery system of FIG. 1 along the line 11-11;

FIG. 3 is a side view of an inner member of the multiple stent delivery system of FIGS. 1 and 2;

FIG. 4 is a partial cross-sectional view of the multiple stent delivery system of FIG. 1 during stent deployment;

FIG. 5 is a partial cross-sectional view of the multiple stent delivery system of FIG. 4 during the process of repositioning where the diameter of the expanded tip of the end of the inner member is slightly narrowed as it is being moved through the inner diameter of the stent as the sheath is advanced;

FIG. 6 is a cross-sectional view of the multiple stent delivery system of FIG. 5 along the line VI-VI;

FIG. 7 is a partial cross-sectional view of the multiple stent delivery system of FIG. 5 after the middle member has been repositioned proximal to the next proximal stent, but before the stent is advanced within the sheath;

FIG. 8 is a schematicized side view of a guidewire member including a tip of a multiple stent delivery system in accordance with another embodiment of the present invention;

FIG. 9 is a partial cross-sectional view of a multiple stent delivery system using the guidewire member and tip of FIG. 8 during deployment of a stent in accordance with one embodiment of the present invention; and

FIG. 10 is a perspective view of an inner member in accordance with another embodiment.

In the following description, the same or similar elements are labeled with the same or similar reference numbers.

### DETAILED DESCRIPTION

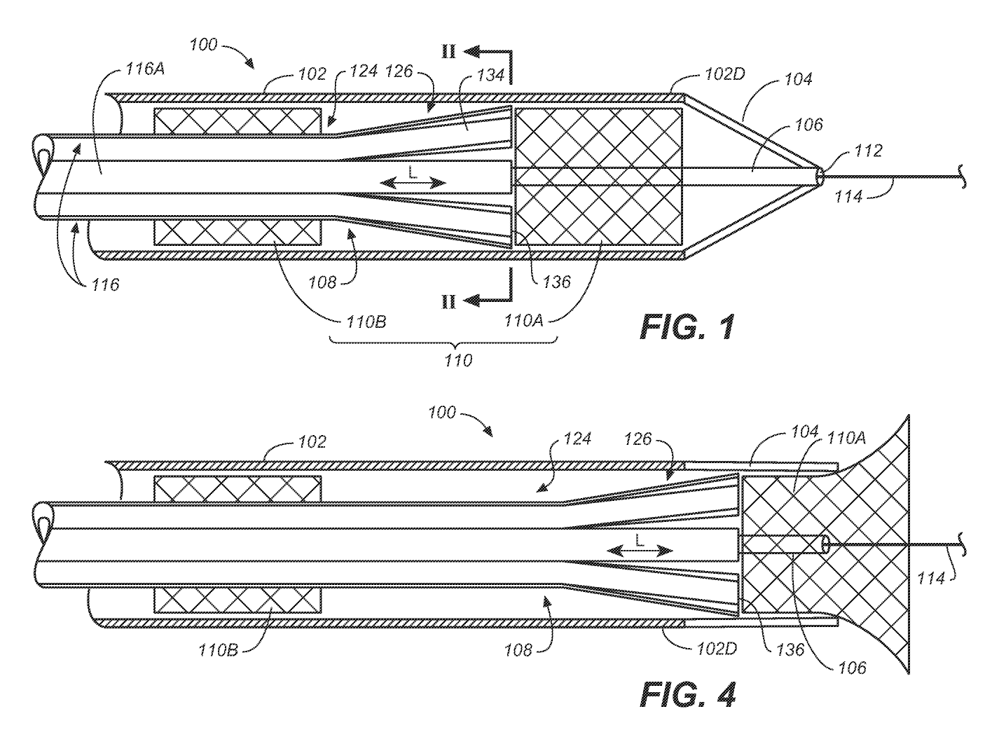

Referring to FIG. 1, a method of deploying multiple stents using a multiple stent delivery system includes moving a distal stent 110A into contact with a stent-pushing face 136 of a compressible expanded tip 126 of an inner member 108. Referring to FIG. 4, a sheath 102 is retracted relative to inner member 108 to deploy distal stent 110A by holding the distal stent 110A stationary with inner member 108 as sheath 102 is withdrawn.

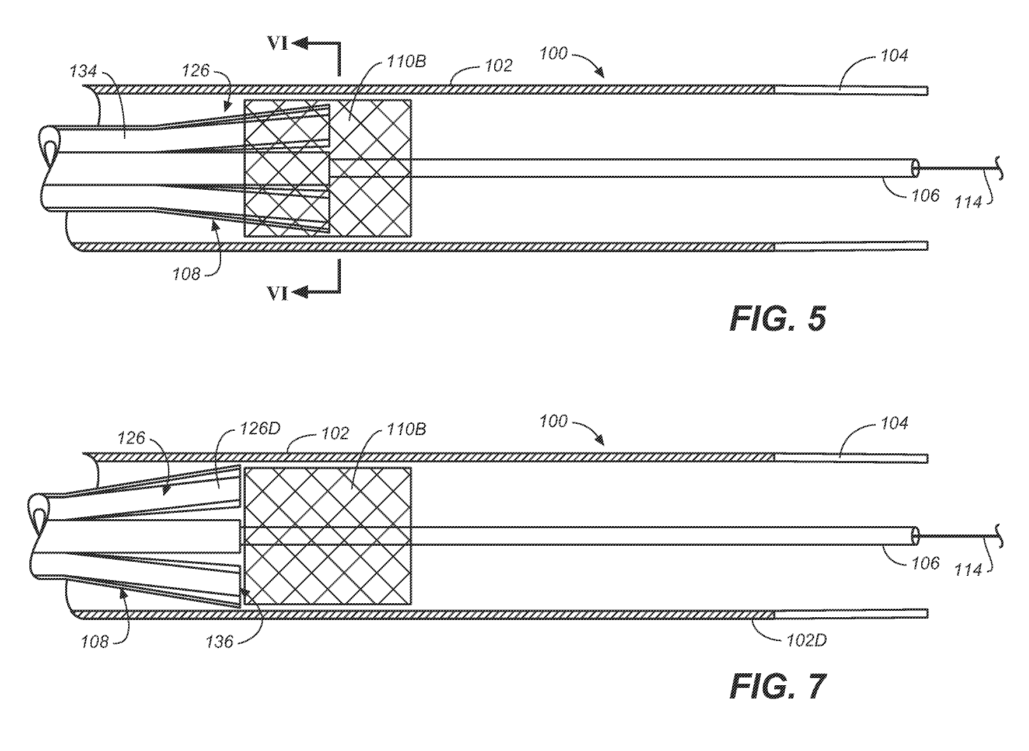

Referring to FIGS. 5 and 7 together, sheath 102 is advanced relative to inner member 108 to reposition a next proximal stent 110B for deployment by passing compressible expanded tip 126 through proximal stent 110B. Sheath 102 is again retracted relative to inner member 108 to engage the proximal stent 110B by pushing proximal stent 110B distally through sheath 102 with inner member 108 to a pre-deployment condition/position, e.g., as shown in FIG. 1. The next proximal stent is then deployed from that position in a manner similar to that illustrated in FIG. 4.

The operations of retracting sheath 102 to deploy a stent (FIG. 4), advancing sheath 102 to capture and reposition the next stent to the pre-deployment position (FIGS. 5, 7), and retracting sheath 102 to deploy the next stent (similar to FIG. 4), can be repeated for any number of stents of multiple stent delivery system 100.

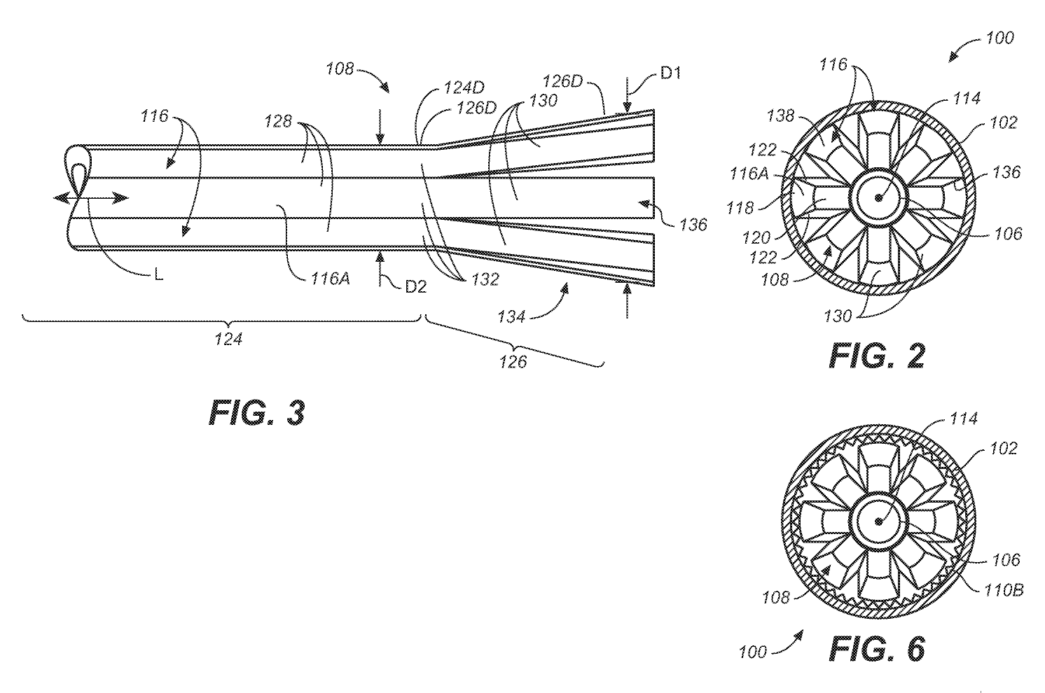

FIG. 1 is a partial cross-sectional view of a multiple stent delivery system 100 in accordance with one embodiment of the present invention. FIG. 2 is a cross-sectional view of multiple stent delivery system 100 of FIG. 1 along the line 11-11.

Referring now to FIGS. 1 and 2 together, multiple stent delivery system 100 includes a sheath 102, a tapered tip 104, a guidewire member 106, an inner member 108, and a plurality of stents 110 including stents 110A and 110B, sometimes called a distal stent 110A and a next proximal stent 110B.

Sheath 102 is a hollow tube and defines a lumen therein through which inner member 108 and guidewire member 106 extend. Sheath 102 includes a distal end 102D.

Tapered tip 104 forms an end position of sheath 102 at distal end 102D of sheath 102. Tapered tip 104 includes a tapered outer surface that gradually increases in diameter. More particularly, the tapered outer surface has a minimum diameter at the distal end of tapered tip 104 and gradually increases in diameter proximally, i.e., in the direction of the operator (or handle of multiple stent delivery system 100), to have a maximum diameter at distal end 102D of sheath 102. Other tip shapes such as bullet-shaped tips could also be used.

Tapered tip 104 is flexible and able to provide trackability in tight and tortuous vessels. Tapered tip 104 includes a guidewire opening 112 therein for connecting to adjacent members and allowing passage of a guidewire 114 through tapered tip 104. Guidewire member 106 extends distally to be adjacent to guidewire opening 112 in tapered tip 104.

As discussed further below, tapered tip 104 is formed of a break open (frangible) construction such that a retraction force causes the tip to press on the stents 110. This force on tapered tip 104 causes tapered tip 104 to break open allowing stents 110 to pass through the petal formation of the now open tapered tip 104.

Stents 110 are self-expanding stents. Stents 110 employ a wire or tube configured (e.g., bent or cut) to provide an outward radial force and employ a suitable elastic material such as stainless steel or Nitinol (nickel-titanium). Nitinol may additionally employ shape memory properties. Although a particular schematic illustration of stents 110 is set forth in the figures, it is to be understood that stents 110 may appear differently in actual implementation depending upon the particular type of stent 110 used.

Stents 110 are radially constrained by sheath 102. More particularly, stents 110 exert an outward radial force on sheath 102. As discussed further below, this outward radial force secures stents 110 to sheath 102 until a sideways force overcomes the friction between the sheath and the stent and causes motion.

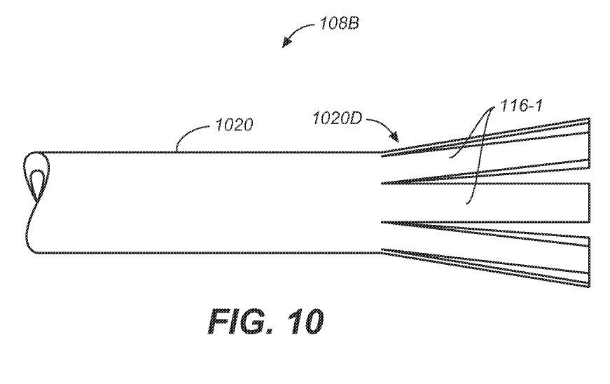

FIG. 3 is a side view of inner member 108 of multiple stent delivery system 100 of FIGS. 1 and 2. Referring now to FIGS. 1, 2 and 3 together, inner member 108 includes a flexible but axially stiff core shaft (constructed for example of metal (such as stainless steel) which is arranged in a spiral or is a hollow tube in which skip cuts have been made to maintain lateral flexibility while providing a high level of axial stiffness, so that there is minimal compressive strain when stents are held stationary. Inner member can be nitinol or a polymer with appropriate structural qualities. Such a structure may include a plurality of splines 116 fixed to the outside of a central core structure similar to that shown in FIG. 10. Such a structure could have splines that only extend through the outer portion of the tubular material and are completely separated and have fingers or other projection of the ends of the splines that radiate outward to form a stent stop face at its distal end. The inner member may include a first spline 116A. In accordance with this example, inner member 108 includes eight splines 116 although can have more or less splines in other examples.

In accordance with this example, splines 116, are an outer layer on an inner core of long trapezoidal or rectangular strips, e.g., of stainless steel or nitinol. Referring to the example illustrated in FIG. 2, splines 116 are trapezoidal in cross-section having a greater width at the outer surface, i.e., the surface adjacent sheath 102, than at the inner surface, i.e., the surface adjacent guidewire member 106, and sides that taper outward from the inner surface to the outer surface. To illustrate, spline 116A includes an outer surface 118 having a greater width than an inner surface 120 of spline 116A. Sides 122 of spline 116A taper outward from inner surface 118 to outer surface 120.

Generally, inner member 108 includes a tubular shaft 124 and a compressible expanded tip 126 formed by spline ends. More particularly, each spline 116 may include a longitudinal runner 128, a finger 130 and an elbow 132, i.e., a bend in spline 116, connecting runner 128 to finger 130. Runners 128 collectively form an outer surface of tubular shaft 124 and fingers 130 collectively form compressible expanded tip 126.

Tubular shaft 124 defines a lumen therein through which guidewire member 106 extend. Tubular shaft 124 includes a distal end 124D and extends proximally from distal end 124D with a substantially uniform diameter D2.

A proximal end 126P of compressible expanded tip 126 of inner member 108 is connected to distal end 124D of tubular shaft 124. Compressible expanded tip 126 defines a tapered outer surface 134 that gradually decreases in diameter. More particularly, tapered outer surface 134 has a maximum first diameter D1 at a distal end 126D of compressible expanded tip 126, i.e., at the distal end of inner member 108, and gradually decreases in diameter proximally, i.e., in the direction of the operator (or handle of multiple stent delivery system 100), to have minimum second diameter D2 at proximal end 126P of compressible expanded tip 126, second diameter D2 being less than first diameter D1.

Compressible expanded tip 126 is formed of outwardly projecting fingers 130, i.e., the distal tips of splines 116. Fingers 130 are self-expanding members and provide an outward radial force on sheath 102. Stated another way, fingers 130 are radially constrained by sheath 102. In one example, splines 116 are bent outwards at elbows 132.

Compressible expanded tip 126 includes an annular stent-pushing face 136 at distal end 126D of expanded tip 126. More particular, each finger 130 includes a planar surface 138 at distal end 126D of expanded tip 126. Planar surfaces 138 are substantially perpendicular to a longitudinal axis L of multiple stent delivery system 100. Planar surfaces 138 collectively defined annular stent-pushing face 136.

Fingers 130 are spaced apart from one another at distal end 126D of expanded tip 126. This spacing allows fingers 130 and thus expanded tip 126 to be radially compressed (move radially inward) during retraction of expanded tip 126 through stents 110. Conversely, annular stent-pushing face 136 and the self-expansion of fingers 130 allow expanded tip 126 to hold stents 110 as sheath 102 is retracted.

FIG. 4 is a partial cross-sectional view of multiple stent delivery system 100 of FIG. 1 during deployment of stent 110A. Referring now to FIG. 4, sheath 102 is retracted relative to inner member 108 to deploy stent 110A.

For simplicity of discussion, motion (retraction and advancement) of sheath 102 shall be set forth herein, however, it is to be understood that retraction or advancement of sheath 102 is simply relative motion of sheath 102 to inner member 108. This relative motion can be accomplished using a variety of techniques, but only the technique where the stent is held stationary and the sheath is retracted is understood to provide a satisfactory result.

Various scenarios include the sheath being retracted while the inner member is held stationary, the sheath being held stationary while the inner member is advanced, and the sheath being retracted and inner member being advanced simultaneously. As used herein, retraction is motion in the proximal direction, i.e., towards the handle or operator, whereas advancement is motion in the distal direction, i.e., away from the operator or handle.

As stents 110 including stent 110A maintain their position within sheath 102 due to the radial self-expanding force exerted by stents 110 on sheath 102, movement of sheath 102 also causes movement of stents 110.

Referring now to stent 110A, stents 110A is retracted until stent 110A comes into contact with stent-pushing face 136 of expanded tip 126 of inner member 108. The contact of stent 110A with expanded tip 126 prevents further retraction of stent 110A while sheath 102 continues to be retracted. The distal force applied to stent 110A by expanded tip 126 becomes greater than the frictional force between stent 110A and sheath 102. Accordingly, stent 110A is pushed distally through sheath 102 by expanded tip 126 and, generally, by inner member 108.

Stent 110A is pushed distally against tip 104, which breaks open as illustrated in FIG. 4. Sheath 102 is retracted until stent 110A while it is being held stationary emerges entirely out from sheath 102 and tip 104 and is thereby deployed in the body lumen.

FIG. 5 is a partial cross-sectional view of multiple stent delivery system 100 of FIG. 4 during repositioning for deployment of stent 110B. FIG. 6 is a cross-sectional view of multiple stent delivery system 100 of FIG. 5 along the line VI-VI. Referring now to FIGS. 5 and 6 together, sheath 102 is advanced relative to inner member 108 to reposition stent 110B to a pre-deployment position. More particularly, from the pre-deployment position (as shown in FIG. 4) as the sheath 102 is again retracted, the stent 110B while being held stationary by the middle member, contacts tapered outer surface 134 of expanded tip 126. However, as the interference force attachment of stent 110B to sheath 102 is greater than the frictional resistance force of tapered outer surface 134 on stent 110B, stent 110B remains attached to sheath 102. Accordingly, the contact of stent 110B on tapered outer surface 134 radially compresses the expanded tip 126. More particularly, fingers 130 are moved radially inwards and closer together.

FIG. 7 is a partial cross-sectional view of multiple stent delivery system 100 of FIG. 5 after repositioning before moving to a pre-deployment position prior to deployment of stent 110B. As distal end 126D of expanded tip 126 moves proximally past stent 110B, distal end 126D of expanded tip 126 self expands into sheath 102. At this stage, sheath 102 is retracted to move the stent to a pre-deployment position near the end of the stent, from this pre-deployment position the sheath 102 is retracted while the inner member is held stationary to deploy stent 110B in a manner similar to that discussed above regarding deployment of stent 110A and FIG. 4.

The operations of retracting sheath 102 to deploy a stent 110, advancing sheath 102 to reposition the next stent 110, retracting sheath 102 to the pre-deployment location/position and deploying the next stent. This process can be repeated for any number of stents 110.

In accordance with this example, stents 110 are relatively short, e.g., 75 mm or less. Short stents require less capital investment for manufacturing equipment than long stents thus decreasing the cost of manufacturing the short stents.

Further, short stents require less material and labor to manufacture than long stents. Thus, in the event that a short stent is defective, the cost of scrapping the short stent is less than the cost of scrapping a long stent.

Further, the physician must overcome a lesser amount of delivery force due to the lesser radial force of the short stent on the sheath, e.g., sheath 102, constraining the short stent than in the case of a long stent. This make placement of the short stent more accurate.

In addition, the lesser amount of frictional force of the short stent on the sheath allows the delivery profile of multiple stent delivery system 100 to be minimized. Minimizing the delivery profile maximizes the anatomical variation in which multiple stent delivery system 100 can be used.

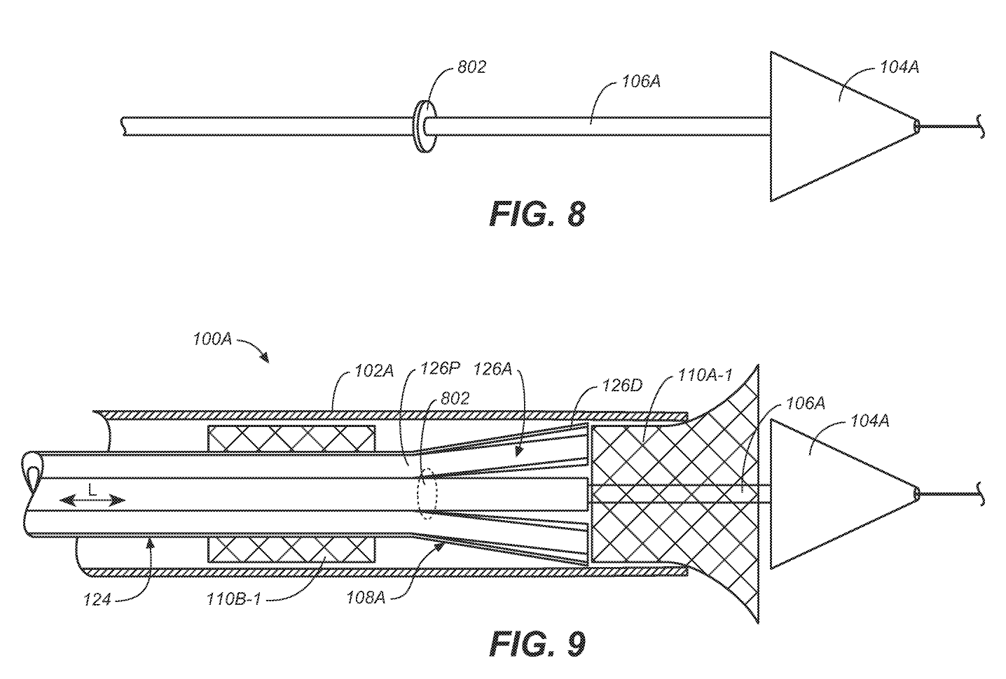

FIG. 8 is a schematic side view of a guidewire member 106A including a tip 104A of a multiple stent delivery system in accordance with another embodiment of the present invention. FIG. 9 is a partial cross-sectional view of a multiple stent delivery system 100A using guidewire member 106A and tip 104A of FIG. 8 during deployment of a stent 110A-1 in accordance with one embodiment of the present invention. Multiple stent delivery system 100A of FIG. 9 is similar to multiple stent delivery system 100 of FIG. 1 and only the significant differences are set forth below.

Referring now to FIGS. 8 and 9 together, in accordance with this example, tip 104A is mounted to the distal end of guidewire member 106A. Further, guidewire member 106A includes a tip stop 802, e.g., an annular disk substantially perpendicular to the longitudinal axis L of guidewire member 106A.

Tip stop 802 can be a disk which contributes to the expanding force within an expanded tip 126A of an inner member 108A during retraction of a sheath 102A such that sheath 102A is retracted relative to both inner member 108A, guidewire member 106A and tip 104A. Thus, as shown in FIG. 9, stent 110A-1 is deployed between a distal end 126D of expanded tip 126A and tip 104A.

However, during advancement of sheath 102A, sheath 102A contacts tip 104A thus releasing tip stop 802 from expanded tip 126A of inner member 108A. After contact with tip 104A, further advancement of sheath 102A also advances tip 104A allowing another stent 110B-1 to be advanced over expanded tip 126A of inner member 108A and thus repositioned for deployment.

More particularly, expanded tip 126A of inner member 108A has a first inner diameter at a distal end 126D of expanded tip 126A and a smaller second inner diameter at a proximal end 126P, and the inner diameter gradually decreases (tapers) between distal end 126D and proximal end 126P. Tip stop 802 has an outer diameter greater than the smaller second inner diameter at proximal end 126P yet less than the first inner diameter at distal end 126D. Accordingly, tip stop 802 slides proximally into expanded tip 126A only until tip stop 802 reaches a point where the inner diameter of expanded tip 126A becomes equal to or less than the outer diameter of tip stop 802, at which point tip stop 802 engages expanded tip 126A. However, tip stop 802 distally slides (releases) from expanded tip 126A freely.

FIG. 10 is a perspective view of an inner member 108B in accordance with another embodiment. Inner member 108B includes a plurality of splines 116-1 that define fingers (similar to fingers 130 of FIG. 3) protruding from the distal end 1020D of a central core structure 1020.

Central core structure 1020 can be a flexible but axially stiff core shaft (constructed for example of metal (such as stainless steel) which is arranged in a spiral or is a hollow tube in which skip cuts have been made to maintain lateral flexibility while providing a high level of axial stiffness, so that there is minimal compressive strain when stents are held stationary. Central core structure 1020 can be nitinol or a polymer with appropriate structural qualities.

The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A multiple stent delivery system comprising:
an inner member comprising a plurality of splines, each of said splines including a longitudinal runner, a finger, and an elbow connecting said runner to said finger, said fingers collectively forming a compressible expanded tip of said inner member.

2. The multiple stent delivery system of claim 1 wherein said elbow is a bend in said spline.

3. The multiple stent delivery system of claim 1 wherein said runners collectively form a tubular shaft of said inner member.

4. The multiple stent delivery system of claim 3 further comprising a guidewire member, wherein said tubular shaft comprises a lumen therein through which said guidewire member extends.

5. The multiple stent delivery system of claim 3 wherein said tubular shaft has a substantially uniform diameter.

6. The multiple stent delivery system of claim 3 wherein a proximal end of said compressible expanded tip of said inner member is connected to a distal end of said tubular shaft.

7. The multiple stent delivery system of claim 6 wherein said compressible expanded tip defines a tapered outer surface.

8. The multiple stent delivery system of claim 6 wherein said compressible expanded tip has a first diameter at a distal end of said compressible expanded tip and a second diameter at said proximal end of said compressible expanded tip, said first diameter being greater than said second diameter.

9. The multiple stent delivery system of claim 1 further comprising a sheath comprising a lumen therein in which said inner member is located, wherein said fingers are self-expanding members and provide an outward radial force on said sheath.

10. The multiple stent delivery system of claim 1 wherein said compressible expanded tip further comprises an annular stent-stop face at a distal end of said compressible expanded tip.

11. The multiple stent delivery system of claim 10 wherein each of said fingers comprises a planar surface perpendicular to a longitudinal axis of said multiple stent delivery system, said planar surfaces collectively forming said annular stent-stop face.

12. The multiple stent delivery system of claim 1 wherein said fingers are spaced apart from one another at a distal end of said compressible expanded tip allowing said fingers to be radially compressed during retraction of said compressible expanded tip through a stent of said multiple stent delivery system.

13. A multiple stent delivery system comprising:
a sheath comprising a lumen therein;
a tip mounted to said sheath;
a plurality of self-expanding stents radially constrained by said sheath; and
an inner member located within said lumen of said sheath, said inner member comprising a plurality of splines, each of said splines including a longitudinal runner, a finger, and an elbow connecting said runner to said finger, said fingers collectively forming a compressible expanded tip of said inner member.

14. The multiple stent delivery system of claim 13 wherein said tip comprises a guidewire opening, said multiple stent delivery system further comprising:
a guidewire member; and
a guidewire passing though said guidewire member and through said guidewire opening of said tip.

15. A multiple stent delivery system comprising:
a sheath comprising a lumen therein;
a plurality of self-expanding stents radially constrained by said sheath;
an inner member located within said lumen of said sheath, said inner member comprising a plurality of splines, each of said splines including a longitudinal runner, a finger, and an elbow connecting said runner to said finger, said fingers collectively forming a compressible expanded tip of said inner member;
a guidewire member extending through said lumen of said sheath, said guidewire member comprising a tip stop for selectively engaging said inner member; and
a tip mounted to a distal end of said guidewire member.

16. A method of deploying multiple stents using a multiple stent delivery system comprising:
moving a distal stent into contact with a stent-stop face of a compressible expanded tip of an inner member;
retracting a sheath relative to said inner member to deploy said distal stent comprising holding said distal stent stationary with said inner member, while retracting said sheath;
advancing said sheath relative to said inner member to reposition a proximal stent for deployment comprising compressing and passing said compressible expanded tip through said proximal stent; and
retracting said sheath relative to said inner member to deploy said proximal stent comprising holding said proximal stent stationary with said inner member while retracting said sheath.

17. The method of claim 16 wherein said moving comprises retracting said sheath relative to said inner member, said distal stent being interferingly attached to said sheath by force between said distal stent and said sheath.

18. The method of claim 17 wherein a distal force applied to said distal stent by said compressible expanded tip of said inner member during said retracting a sheath relative to said inner member to deploy said distal stent is greater than a frictional resistance force associated with the force between said distal stent and said sheath.

19. The method of claim 16 wherein said advancing said sheath relative to said inner member to reposition a proximal stent for deployment comprises contacting a tapered outer surface of said compressible expanded tip against a next proximal stent.

20. The method of claim 19 wherein said contacting comprises radially compressing said compressible expanded tip.

21. The method of claim 20 wherein said fingers of said compressible expanded tip are moved radially inwards and closer together during said radially compressing said compressible expanded tip.

22. The method of claim 19 wherein during said contacting, said next proximal stent is attached to said sheath by interfering forces between said next proximal stent and said sheath, said frictional resistance force between said distal stent and said sheath being greater than a frictional resistance force between said tapered outer surface and said next proximal stent.

23. The method of claim 16 wherein said advancing said sheath relative to said inner member to reposition a proximal stent for deployment further comprises moving a distal end of said compressible expanded tip proximally past said proximal stent.

24. The method of claim 23 wherein said distal end of said compressible expanded tip self-expands into said sheath upon passing said proximal stent.

25. The method of claim 16 wherein said retracting a sheath relative to said inner member to deploy said proximal stent comprising retracting said sheath relative to a tip attached to a guidewire member.

26. The method of claim 25 wherein said guidewire member is selectively attached to said inner member by a tip stop of said guidewire member.

27. A multiple stent delivery system comprising:
an inner member comprising a central core structure and fingers protruding from said central core structure, said fingers collectively forming a compressible expanded tip of said inner member.

28. A multiple stent delivery system comprising:
a sheath comprising a lumen therein;
a tip mounted to said sheath;
a plurality of self-expanding stents radially constrained by said sheath; and
an inner member located within said lumen of said sheath, said inner member comprising a central core structure and fingers protruding from said central core structure, said fingers collectively forming a compressible expanded tip of said inner member.

29. A multiple stent delivery system comprising:
a sheath comprising a lumen therein;
a plurality of self-expanding stents radially constrained by said sheath;
an inner member located within said lumen of said sheath, said inner member comprising a central core structure and fingers protruding from said central core structure, said fingers collectively forming a compressible expanded tip of said inner member;
a guidewire member extending through said lumen of said sheath, said guidewire member comprising a tip stop for selectively engaging said inner member; and
a tip mounted to a distal end of said guidewire member.
